(19) United States
(12) Patent Application Publication
(10) Pub. No.: US 2026/0083409 A1
(43) Pub. Date: Mar. 26, 2026

(54) METHOD FOR DETECTING A SITUATION IN WHICH A PERSON SUFFERS A MEDICAL EMERGENCY, AND FIRST AID SYSTEM

(71) Applicant: MERCEDES-BENZ GROUP AG, Stuttgart (DE)

(72) Inventors: Arnulf FRISCH, Weil Im Schoenbuch (DE); Jürgen SCHMIDT, Köngen (DE); Frank ZAMPONI, Mainz (DE); Arne BECKER, Aachen (DE); Frederik HIRSCH, Aachen (DE); Gereon ULLMANN, Gerlingen (DE); Mark VERJANS, Aachen (DE); Michael KRIEGER, Ingolstadt (DE)

(21) Appl. No.: 18/849,067

(22) PCT Filed: Mar. 6, 2023
(86) PCT No.: PCT/EP2023/055528

§ 371 Date: Sep. 20, 2024

(30) Foreign Application Priority Data
Mar. 22, 2022 (DE) 10 2022 000 984.6

(51) Int. Cl. A61B 5/00; B60Q 9/00; B60R 16/023; H04W 4/40

(52) U.S. Cl. CPC A61B 5/747; A61B 5/6893; A61B 5/746; B60R 16/0231; H04W 4/40; B60Q 9/00

(57) ABSTRACT

A situation in which a person located in a vehicle suffers a medical emergency is detected and aid measures are provided. Vital data is examined for deviations from predefined standard values. If a deviation be identified an alert is output in the vehicle. A communications link is established to an aider external to the vehicle the aider is granted access to at least some of the vital data and to the control of vehicle functions. A microphone and a loudspeaker are activated to establish a bidirectional verbal communications link between vehicle occupants and aider and a camera, which captures at least a portion of a vehicle interior, to transfer camera images to the aider. The aider can talk to the vehicle occupants, visually examine the person, and propose suitable aid measures. The aider controls at least one vehicle function in order to facilitate the examination and/or assist the person.

### BACKGROUND AND SUMMARY OF THE INVENTION

Exemplary embodiments of the invention relate to a method for detecting a situation in which a person located in a vehicle suffers a medical emergency and for providing assistance, as well as to a first aid system.

Following a road traffic accident, rapid action is needed in order to rescue those people involved. In the European Union, any vehicle newly registered after 31 Mar. 2018 must be fitted with the so-called eCall system. This is an automatic emergency call system for motor vehicles that is able to detect an accident and automatically inform the emergency services about it. This means that the emergency services can be dispatched to the site of the accident particularly rapidly by shortening the rescue chain and quickly establishing contact. Via eCall, an alarm can be triggered automatically or also manually. In the process, a data set is transmitted from the vehicle to an emergency call center, the data set containing at least the time of the accident, the GPS coordinates of the accident location, and the direction of travel. Optionally, data, such as the severity of the accident, can also be transmitted from an onboard safety system. In addition, a speech channel is opened to the emergency call center, so that, should the vehicle occupants still be conscious, verbal communication with the emergency services can also take place.

Another emergency assistance system for car accidents is also known from DE 199 45 147 A1. The emergency assistance system can comprise a person injury detector and a first aid device. The person injury detector is able to monitor vital signs of vehicle occupants and assess the extent of the injuries to the casualties following an accident. Depending on the severity of the injuries, an automated system in the vehicle outputs information on how to carry out suitable first aid measures.

Appropriate information can be output acoustically, and a robot voice can provide the casualty with words of encouragement, which is intended to help the casualty remain conscious. The emergency assistance system can also actuate visual and acoustic devices in the vehicle to make people located in the vicinity of the affected vehicle aware of the accident and prompt them to provide first aid.

DE 10 2019 219 498 A1 discloses a method and system for telemedicine treatment in a vehicle. If an impaired state of health of a vehicle occupant is detected, the vehicle occupant is supplied with a questionnaire which they must fill in. The vehicle occupant can forward this questionnaire to a doctor, so that the doctor can assess the state of health of the vehicle occupant more quickly. To this end, the vehicle occupant can manually initiate a telemedicine examination by a doctor. The vehicle can also assess the state of health of the vehicle occupant and, if an emergency condition is determined, automatically inform a doctor about the onset of the emergency situation, activate an automated driving mode to a medical practice and/or retrieve navigation data on how to get to the closest medical practice and configure this for a navigation system.

DE 10 2015 105 581 A1 discloses a system and a method for monitoring the state of health of a vehicle occupant. The system can perform such a detailed assessment of the state of health that an aider can be contacted as soon as a medical emergency first presents itself but is not yet acute. The system can automatically initiate a telemedicine examination. The system can also automatically control vehicle functions such as the air-conditioning unit, lights, or window regulators. To assess the state of health, the system compares a vehicle occupant's vital parameters with permanently defined default values.

DE 10 2012 200 189 A1 discloses a method for operating a vehicle in which the state of health of a driver is determined and on the basis of the result, a safety program to operate the vehicle is activated.

DE 10 2019 211 588 A1 discloses a method and an apparatus for caring for a vehicle occupant in an emergency situation. In this case, vital parameter-specific value ranges are permanently defined for different vital parameters, on the basis of which ranges an escalation level is determined. Depending on the escalation level, different assistance measures are launched, beginning with outputting assistance instructions and an alarm, and escalating to automatically contacting a doctor.

DE 10 2016 207 447 A1 discloses a method and a device for occupant assistance for a vehicle. On the basis of a measured vital parameter, the device can change the ambient conditions in the vehicle interior. In addition, a doctor can transmit therapy instructions to the vehicle, whereby the seat heating of the vehicle occupant's seat is manually controlled from outside of the vehicle in order to treat the vehicle occupant with heat in a targeted manner.

DE 10 2012 014 717 A1 discloses a method and driver assistance system for operating a vehicle if the driver experiences an adverse health event. If there is a medical issue, a communications link to a hospital can be established. The vehicle can receive control commands from the hospital to control devices integrated in the vehicle, for example a seat adjustment. This allows a doctor to treat the driver remotely.

In addition, DE 10 2019 215 351 A1 discloses an emergency information system for a vehicle and a method for operating same. In an emergency, the vehicle can be brought to a standstill automatically and information can be output graphically to potential aiders.

Such emergency systems can be used to improve the initiation of assistance for traffic accident casualties. However, emergency situations also arise in connection with vehicles that do not involve a traffic accident. As life expectancy increases in first world and developed countries, the number of elderly people is also rising. Accordingly, more and more vehicles are being operated by elderly people. However, the risk of illness also increases with age, with cardiovascular system disorders in particular being one of the most frequent causes of death in the western world. For example, a person driving a vehicle might suffer a heart attack or another health-related circulatory collapse while driving their vehicle. To further increase safety in everyday road traffic, there is thus a need for assistance systems which also proactively enable and/or facilitate the provision of first aid in connection with health-related emergencies.

According to the invention, this problem is solved by a method for detecting a situation in which a person located in a vehicle suffers a medical emergency and for providing assistance and by a first aid system.

In a method for detecting a situation in which a person located in a vehicle suffers a medical emergency and for providing assistance, at least one sensor is used to detect vital signs of the person, sensor data generated by the sensor is evaluated by a computing unit to determine vital data and based on the vital data in the vehicle, appropriate instructions for providing assistance to the person in a respective situation are output. The method further provides that the computing unit examines the vital data for deviations from predefined standard values, with person-specific standard values being established which the computing unit takes into account when examining the vital data for deviations, and the computing unit, on identifying a deviation, causes an alarm to be output in the vehicle, wherein, if this is not proactively prevented by a vehicle occupant, the computing unit establishes a communications link to an aider external to the vehicle by means of a telecommunications unit and grants the aider access to at least some of the vital data and access to the control of vehicle functions, wherein the computing unit activates a microphone and a loudspeaker, for the purpose of establishing a bidirectional verbal communications link between vehicle occupants and the aider, and a camera that captures at least a portion of a vehicle interior for the purpose of transferring camera images to the aider, whereby the aider is then able to talk to the vehicle occupants, to visually examine the person and to propose suitable measures for aiding the person on the basis of an examination result, whereby the aider controls at least one vehicle function in order to facilitate the examination and/or to aid the person.

The method according to the invention is based on the idea of monitoring the state of health of vehicle occupants (or at least of the person driving the vehicle) when the vehicle is in use and thereby identifying the onset of medical emergencies, which at a minimum impair safe operation of the vehicle, at an early stage, so that measures can be taken before the ability of the person driving the vehicle to safely control the vehicle has deteriorated so much that this is no longer possible. Accordingly, the state of health of other vehicle occupants can also be monitored, so that emergencies such as the onset of a circulatory collapse or a heart attack can be identified at an early stage and appropriate countermeasures can be initiated at an early stage.

According to the invention, a communications link is established with the aider external to the vehicle, for example a paramedic, tele-emergency doctor, or other suitable medical personnel, who can evaluate the transmitted vital data and examine the person experiencing the medical emergency by using the possibility of bidirectional verbal communication and visual examination via the camera images generated by the camera. Compared to pure sensor-based health analysis by a machine, this examination by a person enables a particularly comprehensive and differentiated assessment of the state of health of the person in need. This makes it possible to determine suitable measures for initiating help in an even more targeted manner.

Because the aider also gains control over vehicle functions by way of the computing unit, the aider is also able to provide help to the person particularly rapidly or to change the vehicle configuration, thereby making it easier for the aider to carry out the examination.

A variety of vital signs of the person can be detected, such as blood pressure, heart rate, breathing rate, skin conductance, skin surface temperature, breathing depth, and the like. Person-specific standard values or standard ranges are established for the detected parameters and/or variables derived therefrom, which do not deviate from the corresponding vital signs in a healthy person. The person-specific standard values or standard ranges can be established based on age, sex, height, weight, pre-existing conditions, etc. If at least one of the vital parameters deviates from the corresponding standard value or standard range, the computing unit is able to assess and establish the nature of the medical emergency. Preferably, the computing unit can detect such vital signs and determine vital data that make it possible to assess a state of the cardiovascular system of the person. This makes it possible to identify an impending heart attack and initiate appropriate assistance before the heart attack occurs.

Preferably, the aider controls at least one of the following vehicle functions:
adjusting a seat;
switching interior lighting on or off;
adjusting a vehicle AC unit;
actuating a window regulator; and/or
adjusting seat heating.

By adjusting the seat, the aider is able to move the person involved in the medical emergency in the vehicle and thus put them into a position having a positive effect on the person's health. In addition, the person can be moved relative to the camera, which makes it easier for the aider to visually examine the person. This is further supported by the aider also being able to activate or deactivate interior lighting in a targeted manner. For example, the aider can switch on lights recessed in the vehicle headliner, which enables a particularly comprehensive visual analysis of the person in need. Actuating a window regulator and/or the vehicle AC unit allows the interior climate in the vehicle to be influenced. For instance, the person experiencing the medical emergency can be supplied with fresh air for example and/or the temperature in the vehicle interior can be raised or lowered. The air mass flow rate conveyed by the vehicle AC unit can also be adjusted. The vehicle AC unit can also be set to recirculate air, which prevents noxious vapors from being drawn in from the vehicle environment. If the person is at risk of getting cold, the seat heating in their vehicle seat can be activated to warm the person up.

To avoid false alarms, an output alarm can also be manually deactivated by a vehicle occupant before the aider is notified. However, if there is no deactivation, be it because there actually is a medical emergency and/or because the person driving the vehicle is travelling alone in the vehicle and is no longer able to input data independently via an appropriate human-machine interface, then the aider is automatically notified by the computing unit.

In addition to the vital data, further data can be transferred to the aider.

This includes, for example, personal data, such as the name, address, age, sex, height, weight, blood group, pre-existing conditions, allergies, implants, illnesses, the wearing of implants, or the like of the person experiencing the medical emergency, and/or vehicle data, which includes all information acquired by the vehicle using sensors and/or information processed in a computing unit. This information includes, for example, a past or present speed of travel of the vehicle, acceleration values, a force exerted by the person driving the vehicle on an accelerator or brake pedal, devices currently active in the vehicle such as an active windscreen wiper, an activated hazard warning light, activated vehicle headlights, a setting of the vehicle AC unit, and the like. This helps the aider make a particularly comprehensive assessment of the respective situation. For example, the operating behavior of the person experiencing the medical emergency, such as a particularly violent shaking of the steering wheel of the vehicle, allows conclusions to be made about particular health problems, for example the occurrence of an epileptic seizure.

Any sensors can be used to monitor the vital signs of the vehicle occupants, in particular of the person driving the vehicle. In accordance with a further advantageous embodiment of the method, at least one vehicle-integrated sensor and/or at least one sensor integrated in a mobile end device worn or carried by the person is used to detect the vital signs. The vehicle-integrated sensor can be, for example, a capacitive sensor for measuring skin conductance, a pressure sensor for measuring movements, the pulse rate, the blood pressure, or the like, a camera, in particular an infrared camera, for measuring the body temperature or the like. Camera images generated by the camera can also be evaluated to establish, for example, certain movement patterns that point to a medical emergency. As a mobile end device, the person experiencing the medical emergency can, for example, carry a smartphone about their person and/or wear a wearable such as a smartwatch or a fitness tracker on their body. A pulse oximeter can also be used to check or determine the blood oxygen saturation.

The aider can verbally instruct the vehicle occupants to perform first aid. If only the person driving the vehicle is in the vehicle, they are thereby able to help themselves. If there are several vehicle occupants in the vehicle, they can provide support. If only the person driving the vehicle is in the vehicle and they have lost consciousness, then the aider can at least provide assistance by actuating the vehicle functions. For even more comprehensive support with carrying out the first aid measures, according to a further advantageous embodiment of the method according to the invention, examination-relevant information and/or the suitable measures for aiding the person are also output visually in the vehicle. The examination-relevant information includes, for example, the vital data acquired by the vehicle. This can be further processed by the aider and, for example, visually enhanced. By way of example, the aider can mark relevant areas in a corresponding pulse rate or ECG plot so that the vehicle occupants can easily understand the corresponding information. If first aid measures are to be taken, for example carrying out cardiac massage or applying a defibrillator, supplementary and instructional information can be output visually in the vehicle. Any display device located in the vehicle can be used for this purpose. The display device can be permanently integrated in the vehicle or be integrated in a mobile end device which has a communications link to the vehicle. By way of example, visual information is displayed on a head unit in the vehicle and/or on a smartphone display screen. This enables the vehicle occupants to interpret the corresponding information particularly rapidly and easily.

A further advantageous embodiment of the method according to the invention furthermore provides that at least some of the vital data is forwarded to third parties, wherein at least some of the vital data is transferred from the vehicle to the aider and from the aider to a central computing unit external to the vehicle, via which third parties are granted access to at least some of the vital data. By providing at least some of the vital data, the third parties can assess the emergency situation that has arisen in the vehicle. Third parties can be, for example, actors involved in the rescue chain, such as an emergency service with an ambulance, an emergency doctor with an emergency doctor vehicle, a hospital, a clinic, a rehabilitation center or the like. By transferring the relevant data, which can also include the personal data and/or vehicle data, to the central computing unit external to the vehicle, this can be made accessible to third parties in particularly simple manner. In this case, the aider can also establish which items of the corresponding data are to be transferred to the central computing unit external to the vehicle and/or which of the third parties has access to which parts of the data. The central computing unit external to the vehicle can be operated in this case, for example, by a vehicle manufacturer or also by an emergency service.

Corresponding data can be transferred in various ways, for example over the internet.

Transfer from the vehicle to the aider is typically effected by mobile radio. However, if appropriate devices are within range, other wired or wireless communication technologies such as WIFI, Bluetooth, or NFC for example can be used.

In accordance with a further advantageous embodiment of the method, the alarm is output in the vehicle haptically, visually, and/or acoustically. This enables a particularly rapid and easy-to-understand interpretation of the alarm, so that the vehicle occupants and/or also persons external to the vehicle are informed of the emergency health situation. For example, actuators integrated into vehicle seats or actuators connected to the steering wheel can be used to output the alarm haptically. For instance, a vehicle seat can vibrate and/or the steering wheel can judder. To visually output the alarm, hazard warning lights disposed in the vehicle interior can be activated and/or warning messages can be displayed on vehicle displays such as the instrument cluster, the head unit and/or another display screen. According to a further advantageous embodiment of the method, the alarm can be visually output external to the vehicle by activating any external vehicle lighting units such as the front headlights, rear lights, brake lights, indicator lights, or the like. These can be controlled in a modulated way, which makes it possible to generate certain light patterns. In particular, illuminated symbols are output which are not output in any other traffic situation. By way of example, a flashing frequency higher than a typical flashing frequency can be selected and/or different lighting devices such as the hazard warning lights and the brake light can be actuated alternately. To acoustically output the alarm, sirens, bells, loudspeakers, or similar warning noises can be output in the vehicle interior and/or a spoken warning message can be output. To sound the alarm external to the vehicle, the vehicle horn can be actuated, for example. In addition, or as an alternative, an external loudspeaker or the like that is mounted on the vehicle can be used to output the alarm. Such an external loudspeaker is already present anyway on an electric vehicle to generate a driving noise.

Preferably, the vehicle is automatically brought to a standstill when the alarm is output. If the person driving the vehicle is no longer able to safely operate the vehicle, the vehicle can be safely brought to a standstill automatically as a result. For this purpose, the vehicle can monitor its environment using a range of environment sensors such as cameras, LIDARs, radar sensors, and/or ultrasound sensors and output corresponding control commands for the at least partially automated control of the vehicle's longitudinal and/or lateral control. The vehicle is brought to a standstill in such a way that other road users are not endangered. This means that not only can the vehicle stop, but it can also drive to a safe part of the road, such as a hard shoulder, a parking area or lay-by or an emergency stopping bay. This increases road traffic safety even further, particularly if the person driving the vehicle loses consciousness. Fixed GPS coordinates are typically known for parking areas and lay-bys and emergency stopping bays. In addition, parking areas and lay-bys and emergency stopping bays are safer for first aiders or third-party helpers (rescue service etc.), since they are not exposed to the risk of flowing traffic.

Provision can be made to identify the position of the vehicle by determining the GPS coordinates. The GPS coordinates can be determined by the vehicle itself and/or a mobile end device and sent to the computing unit external to the vehicle via wireless data transfer.

In the case of a first aid system comprising a vehicle, a computing unit, a telecommunications unit, a sensor, a human-machine interface, and a camera, according to the invention the devices listed are configured to carry out a method according to the invention. The vehicle can be any vehicle such as a passenger car, HGV, van, bus, or the like. One or several sensors can be permanently integrated in the vehicle, with the aid of which the vital signs of vehicle occupants, at least of the person driving the vehicle, are monitored. Corresponding vehicle occupants can also carry mobile end devices about their person, such as smartphones, smartwatches, fitness tracker, pulse oximeters, or the like, which enables even more comprehensive detection and monitoring of vital signs. The corresponding devices or sensors transfer information to the computing unit. The latter can also be permanently integrated in the vehicle.

Using the information transmitted by the sensors, the computing unit generates vital data that is examined for deviations from standard values. Corresponding deviations allow conclusions to be made about the future or present occurrence of a medical emergency. In such a case, the computing unit initiates the outputting of a warning. The human-machine interface, for example a touchscreen, can be used to prevent vehicle occupants from carrying out further measures if an alarm is output. This includes, for example, contacting an aider external to the vehicle by means of the telecommunications unit. In this case, a bidirectional verbal communication channel, in particular via mobile radio, is established with the aider external to the vehicle by means of a vehicle-integrated microphone and loudspeaker. Camera images generated by the camera are likewise transferred via the telecommunications unit to the aider, so that they can assess the situation in the vehicle. Several cameras can also be installed in the vehicle, which permit a visual examination of vehicle occupants from several viewing directions. Giving instructions that can be understood particularly intuitively by vehicle occupants for the purpose of providing first aid measures is possible via the visual output of appropriate information in the vehicle. Any display devices, in particular a central onboard display such as the head unit or the like, are suitable for this purpose. Due to the differentiated examination by the aider external to the vehicle, appropriate information is displayed for the respective situation, which enables particularly efficient treatment of the person experiencing the medical emergency.

### BRIEF DESCRIPTION OF THE DRAWING FIGURES

Further advantageous embodiments of the method according to the invention for detecting a situation in which a person located in a vehicle suffers a medical emergency and for providing assistance as well as the first aid system according to the invention also emerge from the exemplary embodiments, which are described in more detail hereinbelow with reference to the figures, in which:

FIG. 1 shows a schematic illustration of the actors involved in carrying out the method according to the invention; and

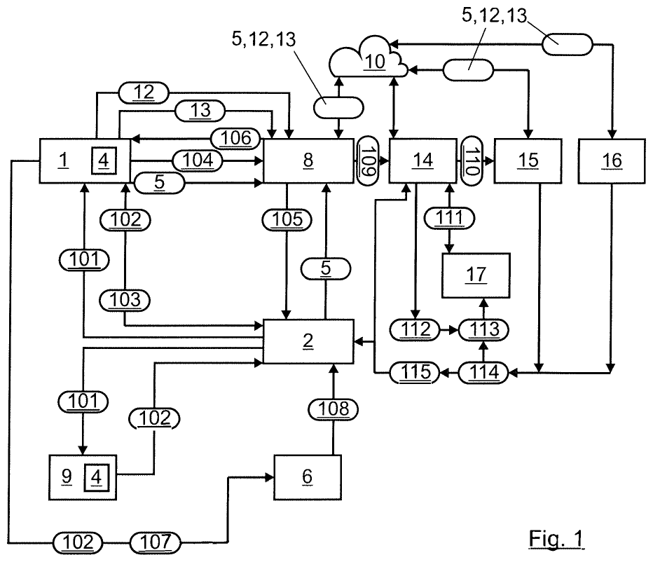

FIG. 2 shows a schematic illustration of the devices involved in carrying out the method according to the invention.

### DETAILED DESCRIPTION

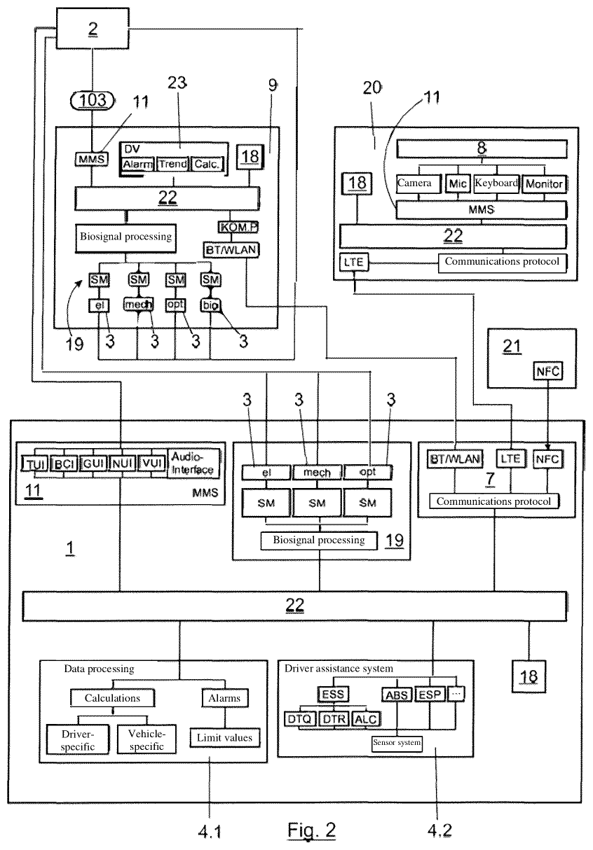

Using the method according to the invention, it is possible to assist a person 2 in a vehicle 1 who is experiencing a medical emergency particularly rapidly and in a targeted manner. The person 2 is monitored for this purpose using at least one sensor 3, shown in FIG. 2, to analyze the vital condition of the person 2. Appropriate sensors 3 can be permanently integrated in the vehicle 1 or in a mobile end device 9. The mobile end device 9 can be carried by the person 2 or worn on the body. The vehicle 1 and/or the mobile end device 9 accordingly collect measurement data 101.

The vehicle 1 and/or the mobile end device 9 comprise a computing unit 4 for evaluating the measurement data 101. The computing unit 4 derives vital data 5 from the measurement data 101, whereby conclusions can be drawn about the state of health of the person 2. If there is at least one deviation in the vital data 5 from pre-defined person-specific standard values, then the corresponding computing unit 4 can cause an alarm 102 to be output in the vehicle 1. In this case, an alarm 102 can be output both via the mobile end device 9 and/or via vehicle-integrated means. By actuating the indicators or tail lights of the vehicle and/or the horn for example, an appropriate alarm 102 can also be output external to the vehicle in order to alert people in the vicinity.

Using the method according to the invention, it is possible to output an alarm 102 to the person 2 before they themselves notice that their health is in immediate danger, such as in the event of an imminent heart attack. As a result of the alarm 102 being output, an aider 8 external to the vehicle is automatically notified about the health emergency, unless this is proactively prevented by the person 2 or another vehicle occupant 6. To this end, the vehicle 1 establishes a communications link 104 to the aider 8 via a telecommunications unit 7 shown in FIG. 2. If the person 2 notices an imminent medical emergency unrelated to their vital signs being monitored, it is also possible for the person 2 to alert the aider 8 by manually inputting an operator control action 103 via a human-machine interface 11, likewise shown in FIG. 2.

If the aider 8 is alerted, then at least some of the vital data 5 is transferred from the vehicle 1 to the aider 8. In addition to at least some of the vital data 5, vehicle data 12 and/or personal data 13 can also be transferred in the process. The vehicle data 12 includes, for example, vehicle-intrinsic data such as a steering angle, a speed of travel, acceleration forces, or the like and the personal data 13 includes, for example, the name, sex, age, weight, height, blood group, and/or the like of the person 2. In addition, bidirectional verbal communication is established between the aider 8 and the person 2 or the vehicle occupants 6 via the communications link 104. As a result, the aider 8 is able to converse with the person 2 and/or the vehicle occupants 6, and by evaluating the received vital data 5, vehicle data 12 and/or personal data 13, they are able to analyze the current situation of the person 2 experiencing the medical emergency.

The aider 8 thus examines or treats the person 2 in step 105. Accordingly, the aider 8 is able to appraise the state of health of the person 2 and recommend suitable measures for providing assistance. On the one hand, these measures are carried out directly by the person 2 and/or the vehicle occupants 6 and, on the other hand, the aider 8 is able to operate vehicle functions themselves in step 106. For instance, the aider 8 is able to adjust a vehicle seat, for example move the vehicle seat forwards or backwards and/or put a backrest upright or fold it back, to switch interior lighting on or off, to adjust a vehicle AC unit in order to change the temperature in the vehicle interior and/or supply the person 2 with fresh air, to actuate a window regulator, and/or adjust seat heating. As a result, the aider 8 is able to examine the person 2 more easily and reliably and in addition, if necessary, provide direct assistance themselves. In order to instruct the vehicle occupants 6, the aider 8 can cause information 107 to be output via output means in the vehicle 1. For example, the information 107 can be output acoustically via loudspeakers and/or a visually via a display device in the vehicle 1. As a result, the vehicle occupants 6 are promoted to employ the appropriate first aid measures 108 for the current emergency situation. Because a human examines the person 2, a particularly differentiated and comprehensive and therefore targeted examination and treatment of the person 2 is possible. This increases the survival chances of the person 2 involved in the emergency.

The data acquired in connection with the method according to the invention, i.e., the vital data 5, vehicle data 12, and/or personal data 13, can be transferred from the aider 8 to a central computing unit 10, for example a cloud server, also referred to as a backend. From there, the appropriate information can be supplied to third parties. These are, for example, a rescue coordination center 14, an ambulance 15, an emergency doctor 16, and/or a hospital 17. In step 109, the aider 8 can also inform the rescue coordination center 14 directly about the emergency, so that the rescue coordination center 14 can arrange immediate assistance, i.e., alerting the ambulance 15, in step 110. This enables rescuers to arrive at the vehicle 1 in particularly short space of time.

It is also possible for appropriate vital data 5, vehicle data 12, and/or personal data 13 to be obtained from a hospital 17 via the rescue center 14. This takes place in a step 111. In step 112, the rescue center 14 can arrange for the person 2 to be assigned to a designated hospital. An advance appointment 113 is made for this purpose. In step 114, the person 2 is transported by the ambulance 15 to the hospital 17, if necessary including an examination by the emergency doctor 16. A working diagnosis can be made in step 115.

FIG. 2 shows in a schematic illustration the devices involved in carrying out the method according to the invention. The vehicle 1 comprises several computing units 4, of which a first computing unit 4.1 is responsible for a data processing of the vital data 5 and a second computing unit 4.2 is responsible for providing a variety of driver assistance systems. Relevant data such as program codes and/or control parameters can be saved in a physical storage medium 18, which can also be part of the corresponding computing units 4.1 and 4.2. The aforementioned human-machine interface 11 is used for interacting with the person 2 and/or vehicle occupants 6. The vehicle 1 likewise comprises at least one, in this case several, sensors 3. In summary, the sensors 3 are each integrated into a sensor system unit 19 with a respective sensor module, abbreviated as “SM”, and biosignal processing. For example, an electric or electronic sensor 3 “el”, a mechanical sensor 3 “mech”, and/or an optical sensor 3 “opt” can be used as sensors 3.

The telecommunications unit 7 is used to communicate with the mobile end device 9, a remote assistance system 20, via which the aider 8 can be reached, and optionally an electronic health card 21. All customary technologies such as for example Bluetooth, WLAN, in particular Wi-Fi, mobile radio, for example using LTE, and/or NFC can be used as a communication protocol.

The corresponding devices in the vehicle 1 communicate in this case via a data bus 22.

The mobile end device 9 also comprises a human-machine interface 11, a data bus 22, a corresponding sensor system unit 19 and means for wireless data transfer, which use the “KOM. P” communication protocol on which a said radio technology such as Bluetooth or WLAN is based. The mobile end device 9 also comprises a processor 23 for carrying out data processing “DV” and a physical storage medium 18 for storing program codes.

The remote assistance system 20 also comprises means for carrying out wireless communication, such as an LTE interface and a corresponding underlying communication protocol. Data is forwarded within the remote assistance system 20 in the same way via a data bus 22. A physical storage medium 18 and a human-machine interface 11, via which the aider 8 can interact with the person 2 and/or the vehicle occupants 6, are also part of the remote assistance system 20.

The central idea of the invention is to enable a bidirectional verbal communication link between the aider 8 and at least the person 2, as well as the option for the aider 8 to carry out a visual examination of the person 2 with the aid of camera images generated by camera(s) arranged in the vehicle 1. The aider 8 is additionally able to remotely control vehicle functions to facilitate the examination and/or to directly initiate first steps to assist the person 2.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-9. (canceled)

10. A method for detecting a situation in which a person located in a vehicle suffers a medical emergency and for providing assistance, the method comprising:
detecting, by at least one sensor, vital signs of the person;
evaluating, by a computing unit, sensor data generated by the sensor to determine vital data;
outputting, by the computing unit and based the vital data in the vehicle, instructions for providing assistance to the person in the situation;
examining, by the computing unit, the vital data for deviations from standard values;
causing, by the computing unit when the examination identifies the deviations from the standard values, an alarm to be output in the vehicle;
establishing, by the computing unit using a telecommunications unit, a communications link to an aider external to the vehicle;
granting, by the computing unit, the aider access to at least some of the vital data;
activating, by the computing unit, a microphone and a loudspeaker to establish a bidirectional verbal communications link between occupants of the vehicle and the aider; and
capturing, by a camera, at least a portion of a vehicle interior to transfer camera images to the aider, wherein the aider is then able to talk to the vehicle occupants, to visually examine the person, and to propose measures for aiding the person based on an examination result,
wherein person-specific standard values are established that the computing unit takes into account when examining the vital data for the deviations,
wherein the computing unit only establishes the communications link to the aider if establishing the communications link to the aider is not proactively prevented by a vehicle occupant, and
wherein the computing unit grants the aider access to the control of vehicle functions so that the aider controls at least one vehicle function to facilitate the examination or to aid the person.

11. The method of claim 10, wherein the aider controls at least one of the following vehicle functions:
adjusting a seat;
switching interior lighting on or off;
adjusting a vehicle AC unit;
actuating a window regulator; and
adjusting seat heating.

12. The method of claim 10, wherein the detected vital signs and the determined vital data allows assessment of a state of the cardiovascular system of the person.

13. The method of claim 10, wherein the at least one sensor is a vehicle-integrated sensor or is integrated in a mobile end device worn or carried by the person.

14. The method of claim 10, wherein examination-relevant information or the suitable measures for aiding the person are output visually in the vehicle.

15. The method of claim 10, wherein at least some of the vital data is forwarded to third parties, wherein at least some of the vital data is transferred from the vehicle to the aider, and from the aider to a central computing unit external to the vehicle, via which the third parties are granted access to at least some of the vital data.

16. The method of claim 10, wherein the alarm is output in the vehicle haptically, visually, or acoustically.

17. The method of claim 10, wherein
the alarm is also output external to the vehicle visually or acoustically, or
the vehicle is automatically brought to a standstill when the alarm is output.

18. A first aid system for detecting a situation in which a person located in a vehicle suffers a medical emergency and for providing assistance, the first aid system comprising:
a vehicle comprising a computing unit, a telecommunications unit, a human-machine interface, and a camera; and
at least one sensor,
wherein the at least one sensor is configured to detect vital signs of the person,
wherein the computing unit is configured to
evaluate sensor data generated by the sensor to determine vital data;
output, based the vital data in the vehicle, instructions for providing assistance to the person in the situation;
examine the vital data for deviations from standard values;
cause, when the examination identifies the deviations from the standard values, an alarm to be output in the vehicle;
establishes, using the telecommunications unit, a communications link to an aider external to the vehicle;
grants the aider access to at least some of the vital data; and
activate a microphone and a loudspeaker to establish a bidirectional verbal communications link between occupants of the vehicle and the aider;
wherein the camera is configured to capture at least a portion of a vehicle interior to transfer camera images to the aider, wherein the aider is then able to talk to the vehicle occupants, to visually examine the person, and to propose measures for aiding the person based on an examination result,
wherein person-specific standard values are established that the computing unit takes into account when examining the vital data for the deviations,
wherein the computing unit is configured to only establish the communications link to the aider if establishing the communications link to the aider is not proactively prevented by a vehicle occupant, and
wherein the computing unit is configured to grant the aider access to the control of vehicle functions so that the aider controls at least one vehicle function to facilitate the examination or to aid the person.
